(19) United States
(12) Patent Application Publication
(10) Pub. No.: US 2015/0112132 A1
(43) Pub. Date: Apr. 23, 2015

(54) ELONGATE MEDICAL INSTRUMENT WITH SHEATH

(71) Applicant: Veritract, Inc., Salt Lake City, UT (US)

(72) Inventors: Timothy R. Nieman, North Salt Lake, UT (US); Barry K. Hanover, Park City, UT (US)

(21) Appl. No.: 14/403,111

(22) PCT Filed: May 23, 2013
(86) PCT No.: PCT/US2013/042534

§ 371 Date: Nov. 21, 2014

Related U.S. Application Data
(60) Provisional application No. 61/650,800, filed on May 23, 2012.

(51) Int. Cl. A61B 1/00; A61B 1/05; A61B 1/12; A61B 1/06; A61M 25/01; A61B 1/005; A61J 15/00; A61B 1/015

(52) U.S. Cl. CPC A61B 1/00142; A61J 15/0003; A61B 1/051; A61B 1/015; A61B 1/06; A61B 1/00096; A61M 25/0147; A61B 1/0051; A61B 1/126; A61M 2025/0166
USPC 600/109; 600/122

(57) ABSTRACT

An elongate medical instrument that may comprise steering mechanisms, optical sensors, light emitters, and/or fluid flow paths is disclosed. A removable sheath may be utilized to isolate the instrument from contamination when the instrument is used within the human body. The sheath may be disposable or reusable. In some instances, the elongate device may be used to position other components, such as elongate tubes.

### TECHNICAL FIELD

The present disclosure relates generally to medical devices. More specifically, the present disclosure relates to elongate instruments configured for use within the human body. A sheath may be provided in connection with the elongate instrument to isolate the instrument from interaction with the body environment.

### BACKGROUND

Medical instruments may be configured for use in connection with procedures wherein a portion of the device is located within the human body while the remainder of the device is outside the body. Such instruments may include tubes, probes, endoscopes, stylets, feeding tubes, and so forth. Proper placement of such instruments may be necessary for a particular therapy; however, many such instruments are placed without immediate visual confirmation that the instrument is properly located.

A variety of elongate instruments may be configured for introduction into the human body. Some such instruments may be configured to enter the body through orifices in the body and/or may be configured to traverse or follow internal body lumens. Other elongate instruments may be configured to cross bodily structures (through openings created by a trocar or incision, for example).

The current disclosure is relevant to all such medical and elongate instruments, including elongate tubes configured to facilitate access to interior portions of the body. For example, elongate tubes may be configured for use in connection with delivery of drugs, nutrients, water, and/or other substances to interior portions of the body. Specific examples may include tubes configured for use in connection with drug delivery, including chemotherapy drugs. Such tubes may be configured to access portions of the gastrointestinal tract, such as the stomach or small intestine. Some such tubes may be configured for access via the nose or mouth of a patient while other tubes may be configured for introduction through a surgically-created opening in the body, such as through the abdominal wall. In other instances, elongate tubes may access other portions of the body.

Feeding tubes are one example of elongate instruments configured for use within the human body. Notwithstanding any specific examples recited herein, disclosure provided in connection with a specific elongate instrument (such as a feeding tube) may be analogously applied to other elongate instruments.

As an illustrative example, feeding tubes configured for placement in the human body may create complications if misplaced within the body. In some instances, feeding tubes configured to access the body via the nose and/or esophagus (such as NG-type feeding tubes) may not be configured with components that allow a practitioner to visually guide the tube during delivery. Again, however, misplacement of the tube may result in serious complications for the patient. In the case of feeding tubes, the tube may be incorrectly placed in the lungs of the patient. In extreme cases the tube may even be passed into a patient's brain. Both cases may result in serious complications or death.

Specifically, placement of a feeding tube within the lungs or another internal cavity of a patent may result in complications such as punctured lungs or pneumonia. Approximately 1.2 percent of feeding tube placements result in an inadvertently punctured lung. In approximately 0.5 percent of those instances, the patient dies as a result.

Similarly, feeding tubes configured to access the body by crossing bodily structures such as the abdominal wall (such as G- or J-type feeding tubes) may likewise be configured for precise placement within the body. Again, misplacement of such tubes may result in serious complications.

To address these problems, proper placement of an elongate instrument such as a feeding tube may be confirmed through use of x-ray, pH tests, auscultation, or fluoroscopy. However, in many instances, these tests are performed after placement is completed and, thus, do not provide real-time feedback during the placement procedure. In many misplacement cases, damage may already have occurred by the time the placement is checked. Furthermore, these tests may not provide sufficient information to confirm placement. For example, a feeding tube may be checked by x-ray (a two-dimensional image) to confirm the tube is disposed below the diaphragm. In some instances, however, a tube may reach this position by passing through a lung and rest along the inferior aspect of the diaphragm where it may appear to reside below the diaphragm on x-ray. Thus, a two-dimensional image may be insufficient to confirm placement in the gastrointestinal tract. Further, fluoroscopy or x-ray cannot confirm placement of a tube in the small intestine as opposed to the stomach in all cases.

Additionally, these tests may be expensive and may expose a patient to potentially harmful radiation. Moreover, these procedures may be time-consuming to arrange and, thus, may delay the use of the device after placement. Currently, average time from ordering feeding tube placement to the beginning of feeding is from 22 to 26 hours. If the tube is improperly positioned, this time can be even longer. Thus, there may be significant delay in the delivery of nutrients or medications to the patient.

In some procedures, an endoscope may be used to visualize and direct placement of an instrument, such as a feeding tube. For example, an endoscope may be used to position a guidewire, which may then be used to place the tube.

Thus, as endoscopes or similar devices comprising imaging and/or steering components may be expensive, it may be desirable to reuse such devices in multiple treatments. Accordingly, the device must be sterilized and prepped between use in one patient and subsequent use in another patient. The sterilization procedure itself may be costly and time-consuming. For example, it may involve mechanical cleaning, leakage testing, disinfecting through use of chemicals, rinsing, and drying. In many instances, special training is required to complete this procedure.

### BRIEF DESCRIPTION OF THE DRAWINGS

The embodiments disclosed herein will become more fully apparent from the following description and appended claims, taken in conjunction with the accompanying drawings. These drawings depict only exemplary embodiments, which will be described with additional specificity and detail through use of the accompanying drawings in which:

FIG. 1 is a perspective view of an elongate tube assembly.

FIG. 2 is a perspective view of a portion of the stylet of the elongate tube assembly of FIG. 1.

FIG. 3 is an enlarged view of the distal end of the stylet of FIG. 2.

FIG. 4 is a perspective view of the stylet and sheath of the elongate tube assembly of FIG. 1.

FIG. 4A is a perspective view of a first embodiment of a sheath lens.

FIG. 4B is a perspective view of a second embodiment of a sheath lens.

FIG. 4C is a perspective view of a third embodiment of a sheath lens.

FIG. 5 is a perspective view of an elongate tube assembly illustrating fluid flow through the assembly.

FIG. 6A is an enlarged view of the distal end of the elongate tube assembly of FIG. 5 in a first configuration.

FIG. 6B is an enlarged view of the distal end of the elongate tube assembly of FIGS. 5 and 6A in a second configuration.

FIG. 7 is a cross-sectional view of the elongate tube assembly of FIG. 6A taken through plane 7-7.

FIG. 8 is a cross-sectional view of the elongate tube of FIG. 5 taken through plane 8-8.

FIG. 9 is a an enlarged perspective view of a portion of the distal end of another embodiment of an elongate medical instrument assembly.

FIG. 10A is a partial perspective view of a portion of an endoscope.

FIG. 10B is a cross sectional view of the endoscope of FIG. 10A.

FIG. 11 is a partial perspective view of the distal end of an endoscope.

FIG. 12 is a partial perspective view of the endoscope of FIG. 11 with the endoscope cap removed.

FIG. 13 is a perspective view of the imaging component of the endoscope of FIGS. 10A and 10B.

FIG. 14 is another perspective view of the imaging component of FIG. 13.

FIG. 15 is a partial perspective view of the distal end of an endoscope.

FIG. 16 is a partial cut-away view of the endoscope of FIG. 15.

FIG. 17 is partial perspective view of the imaging component of the endoscope of FIG. 15.

FIG. 18 is a perspective view of the light source of the imaging component of FIG. 17.

FIG. 19 is a partial perspective view of the distal end of an endoscope.

FIG. 20 is a partial perspective view of the endoscope of FIG. 19 with the endoscope cap removed.

FIG. 21 is a side view of a camera and sheath.

### DETAILED DESCRIPTION

Elongate medical instruments, such as stylets, endoscopes, and so forth, may be configured for introduction into the human body for a variety of treatments or therapies. For example, a stylet may be configured to introduce and position additional components, such as feeding tubes, within the body. Similarly, endoscopes may be configured for remote access or viewing within the body. Other elongate instruments configured for use in connection with a variety of therapies are within the scope of this disclosure, including instruments for minimally invasive procedures, instruments for use in the vasculature of a patient, instruments configured for use within the gastrointestinal tract of a patient, instruments configured for short or long term delivery or withdrawal of fluids and/or materials, and so forth. Furthermore, instruments configured for introduction into a body structure or lumen (e.g., NG-type feeding or delivery tubes) as well as instruments configured to traverse bodily structures, natural orifices, or stoma (e.g., G-type, J-type, J-extension type feeding or delivery tubes) are within the scope of this disclosure. Notwithstanding any specific examples given below, any feature of the present disclosure may analogously be applicable to other types of instruments.

A sheath may be provided and configured to isolate one or more components of an elongate instrument from the environment within the body when the instrument is in use. For example, a sheath may be configured to isolate a stylet or other instrument from interaction with bodily fluids or tissues and, thus, may obviate the need to sterilize the stylet or instrument after each procedure. The sheath may be disposable or may be configured to be sterilized and reused.

It will be readily understood that the components of the embodiments as generally described and illustrated in the figures herein could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless indicated.

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “proximal” and “distal” are used herein to refer to opposite locations on a medical device. The proximal end of a device is defined as the end closest to the practitioner when the device is being used or manipulated by a practitioner. The distal end is the end opposite the proximal end, along the longitudinal direction of the device, or the end furthest from the practitioner. It is understood that, as used in the art, these terms may have different meanings with regard to devices deployed within the human body (i.e., the “proximal” end may refer to the end closest to the head or heart of the patient depending on the application). For consistency, as used herein, the ends labeled “proximal” and “distal” prior to deployment remain the same regardless of whether the device is disposed within a human body.

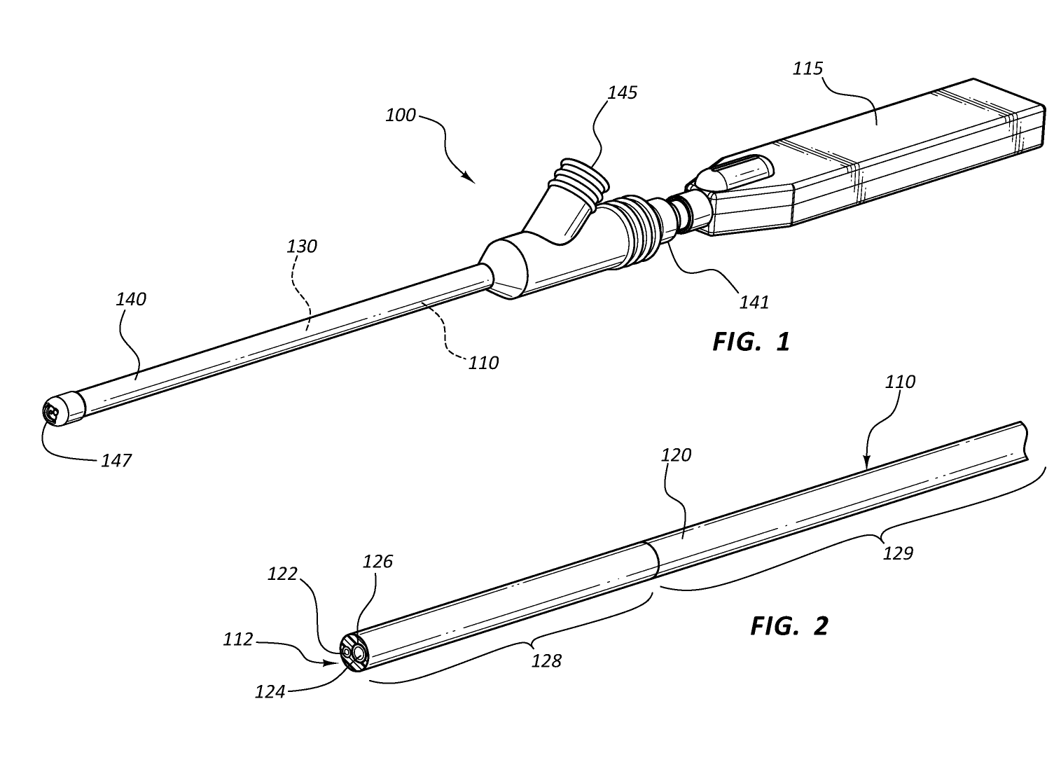

The current disclosure may be applicable to a wide variety of specific elongate instruments, including tubes, such as nasogastric (NG); gastric (G); or jejunostomy (J) feeding tubes, NG, G, GJ, G-J extension, or J drug delivery tubes, other drug delivery tubes, fluid lines, and so forth. FIGS. 1-9 and the accompanying disclosure describe elongate medical devices, which may include elongate tubes. The disclosure included below with respect to “elongate tubes” generally may be applicable to any type of tube described herein or other analogous medical tubes.

FIG. 1 is a perspective view of an elongate tube assembly 100. The elongate tube assembly 100 comprises a stylet 110, a sheath 130, and an elongate tube 140. The stylet 110 comprises an elongate body (120 of FIG. 2) coupled to a connector 115. As further described below, the stylet body 120 may further comprise a variety of subparts, such as optical sensors, light emitting components, steering mechanisms, and so forth. Though the illustrated embodiment includes a stylet 110, other elongate medical instruments may have analogous features, components, or uses. Thus, disclosure provided in connection with the stylet 110 or related components may be applicable to a wide variety of elongate instruments, such as endoscopes, for example. It is within the scope of this disclosure to include any number of features or subcomponents of the stylet 110 in connection with other elongate instruments.

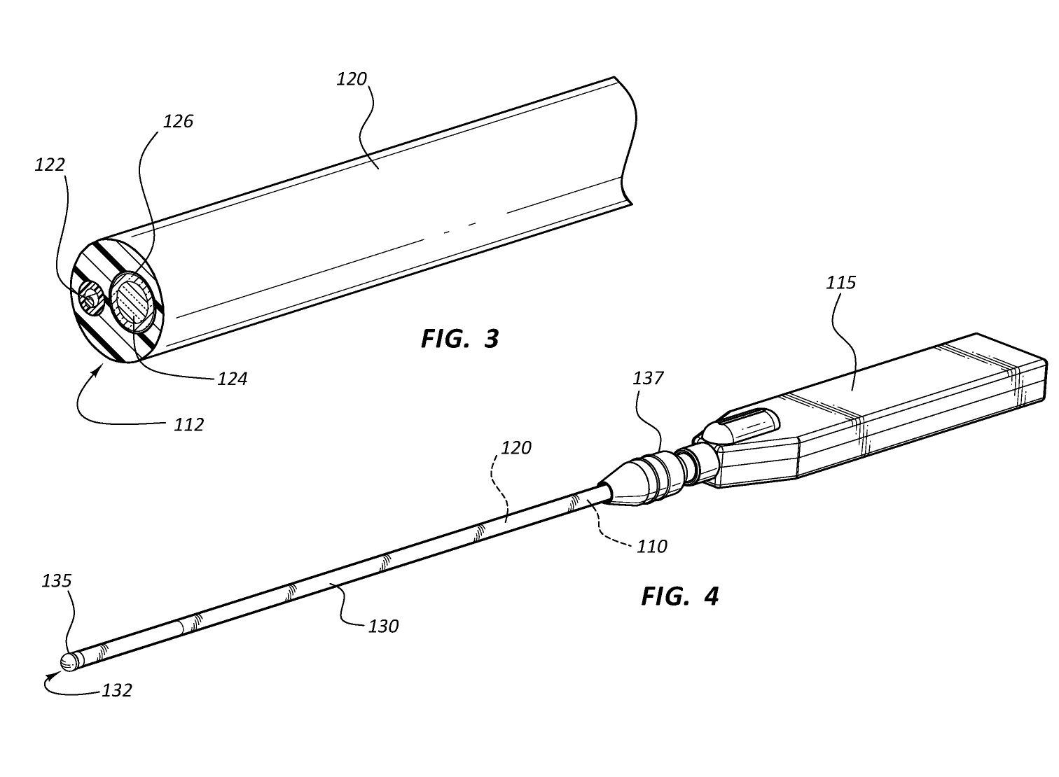

FIG. 2 is a perspective view of a portion of the stylet body 120 of the elongate tube assembly 100 of FIG. 1. As shown in FIG. 2, the stylet body 120 may comprise a steering mechanism such as steering cable 122, an optical sensor 124, and a light emitting component, such as light source 126. These elements are also shown in FIG. 3, which is an enlarged view of the distal end 112 of the stylet body 120 of FIG. 2.

Referring now to FIGS. 1, 2, and 3, the connector 115 may be configured to allow a user to interface with the individual components of the stylet body 120. The connector 115 may be configured to couple to a user interface component, such as a handle. Thus, a user may interface with the connector 115 and, thus, the components of the device, through interface with, or manipulation of, a handle. In some embodiments, a handle may be integral with the connector 115, while in other embodiments, the handle may be a separate component. Still further, it is within the scope of this disclosure for certain interface components to be configured for use through direct interface with the connector 115 while others are configured for use in connection with a handle or other component. Thus, interface or control mechanisms (e.g. image display components, steering input components, and so on) may be described below in connection with the connector 115, a handle, or both. Regardless of any specific example given below, any interface or control mechanism may be provided in connection with the connector directly or in connection with another component, such as handle, configured to be coupled to the connector 115. Examples below referencing user interaction with, or input at, the connector 115 encompass both direct user interaction with the connector 115 and interaction through another component, such as a handle.

The steering cable 122 may be coupled to the stylet body 120 adjacent the distal end 112 of the stylet body 120. The connector 115 may comprise an interface or control configured to allow the user to manipulate the steering cable 122 via the connector 115. In other embodiments, steering mechanisms may comprise multiple cables. For example, the steering mechanism may comprise one, two, three, four, or more cables configured to manipulate the distal end 112 of the stylet body 120 in response to user input at the connector 115, as described below. For example, four separate cables coupled at connection points spaced evenly or substantially evenly around the distal end 112 of the stylet body 120 may be configured to work cooperatively to manipulate the distal end 112 of the stylet body 120. Furthermore, in some embodiments, the user steering interface may be coupled to the connector 115, though not necessarily part of the connector 115.

The stylet body 120 may be comprised of a relatively flexible material configured to bend in response to interaction with the steering cable 122. Thus, a user may be able to direct the distal end 112 of the stylet body 120 through interaction only with the connector 115. In some embodiments, the stylet body 120 may have multiple portions with different characteristics. For example, the stylet body 120 has a distal portion 128 and a proximal portion 129. The distal portion 128 may be comprised of a more flexible material, with respect to the proximal portion 129. Thus, the proximal portion 129 may be configured to be stiffer, thus facilitating advancement of the stylet body 120 while the distal portion 128 is softer, allowing for easier directional maneuverability. In some embodiments, the stylet body 120 may be comprised of an elastomeric material, with the distal portion 128 comprised of a lower durometer material than the proximal portion 129.

Furthermore, the connector 115 may comprise a portion configured to interface with the optical sensor 124. The optical sensor 124 may be configured to transmit images to the connector 115 where the image may be displayed. In some embodiments, the optical sensor 124 may comprise one or more fiber optic strands, configured to transmit an image from the distal end 112 of the stylet body 120 to the connector 115. In other embodiments, the optical sensor 124 may comprise a camera, such as a CMOS or CCD camera, which may convert an image to an electrical signal that may be sent to the connector 115. In some embodiments, a user viewing interface may be positioned on the connector 115; for example, an eyepiece or screen may be mounted to the connector 115. In other embodiments, the connector 115 may comprise a connection, such as an electrical or optical connection, for use with a separate viewing interface, such as an interface coupled to a handle.

Moreover, the stylet body 120 may comprise a light source 126. The light source 126 may be an LED or other light source electrically connected to a power source, such as in the connector 115. In other embodiments, the light source 126 may comprise one or more fiber optic strands configured to transmit light to the distal end 112 of the stylet body 120.

The light source 126 may be configured for use in connection with the optical sensor 124. For example, light from the light source 126 may reflect off structures or elements onto the optical sensor 124. The positioning of the light source 126 may, thus, be configured to aid in viewing via the optical sensor 124 without the light source 126 interfering with or “washing out” the image transmitted by the optical sensor 124. In the illustrated embodiment, the light source 126 comprises a ring of fiber optic strands disposed circumferentially around the optical sensor 124. In other embodiments, the light source 126 may be positioned laterally away along the distal end 112 of the stylet body 120 from the optical sensor 124. In still other embodiments, a light source may additionally or alternatively be provided on a component other than the stylet body 120.

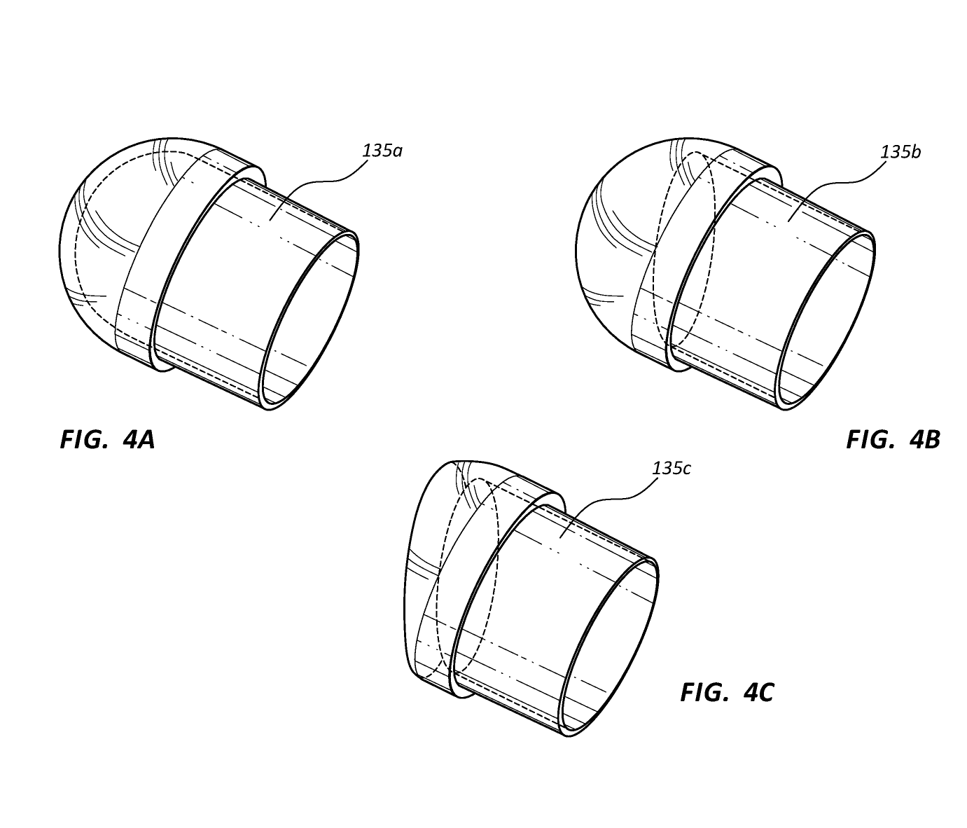

FIG. 4 is a perspective view of the stylet body 120 and sheath 130 of the elongate tube assembly 100 of FIG. 1. Thus, with respect to FIG. 1, in FIG. 4, the elongate tube 140 has been removed, uncovering the sheath 130. The sheath 130 may be configured to seal the body 120 of the stylet 110 such that the body 120 is isolated from contamination, such as by bodily fluids, during use. In other words, the sheath 130 may be configured such that the stylet body 120 may not need to be sterilized before or after use, because the stylet 110 does not come into direct contact with the body environment. Thus, the sheath 130 may be configured as a disposable part, facilitating the quick and easy reuse of the potentially more expensive stylet body 120, including components such as the steering cable, optical sensor, and/or light source (122, 124, and 126 of FIG. 3, respectively) that are associated with the stylet body 120. In other instances, the sheath 130 may be configured to be resterilized and reused, as it may be easier or cheaper to sterilize the sheath 130 as opposed to the stylet body 120.

In some embodiments, the sheath 130 may be elastic or otherwise extensible, allowing a user to stretch or deform the sheath 130. Thus, in some embodiments, the sheath 130 may be configured to stretch tightly over the stylet body 120. Further, in some instances, the sheath 130 may only be elastic in one direction, for example only configured to stretch in the axial direction. In still other embodiments, the sheath 130 may be relatively inelastic.

In some embodiments, the sheath 130 may comprise a lens 135 positioned at or adjacent the distal end 132 of the sheath 130. The lens 135 may be integrally formed with the entire sheath 130 or may comprise a separate component that is coupled to the sheath 130.

The lens 135 may be configured to allow light to pass through the lens 135 without unwanted distortion. For example, in some embodiments, light emitted from the light source (126 of FIG. 3) may pass through or reflect off one or more surfaces of the lens 135, reflect off structures within the body, pass again through the lens 135, and fall on the optical sensor (124 of FIG. 3). Thus, the lens 135 may be shaped or otherwise configured to allow or prevent this reflection and/or similar light interactions while minimizing or controlling the distortion or bending of the light. In some embodiments, the lens 135 may comprise a compliant material, configured to conform to the distal end (112 of FIG. 3) of the stylet body 120. For example, the lens 135 may comprise a thin, compliant film configured to stretch over, and conform to, the distal end (112 of FIG. 3) of the stylet body 120. In other embodiments, as described below, the lens 135 may be formed in a particular geometric shape.

FIGS. 4A, 4B, and 4C are three embodiments of lenses 135a, 135b, 135c for use in connection with a sheath (130 of FIG. 4). As described above, the lenses 135a, 135b, 135c shown in FIGS. 4A-4C may be integrally formed with a sheath, or formed separately and coupled to the sheath. In either case, the lenses may be configured to seal or isolate the interior of the sheath. The lens may be configured with a geometrically shaped interior or exterior surface, which may be configured to control or bend light passing through the lens and/or control or direct reflections of the light source. In the embodiment of FIG. 4A, the lens 135a is configured with a curved outside surface and a curved inside surface. In the embodiment of FIG. 4B, the lens 135b has a flat inner surface and a curved or domed outer surface. In some embodiments, the flat inner surface of the lens 135b may be at a 90 degree angle relative to the longitudinal axis of the lens 135b or stylet body (120 of FIG. 4), or may be at a different angle such as 1-89 degrees. When used in connection with a lens such as lens 135a of FIG. 4A, there may be a gap between a medical instrument having a flat distal end (such as the stylet body 120 of FIG. 2) and the inside surface of the lens 135a. In some embodiments, gaps between the lens (135 of FIG. 4) and the distal end of the instrument (112 of FIG. 2) disposed within the sheath (130 in FIG. 4) may be filled by a coupling fluid, such as silicone gel. Coupling fluids may be configured to influence light transmission across the gap. Other embodiments may not have a gap. For example, the flat inner surface of the lens 135b of FIG. 4B may be configured to abut the distal end of a medical instrument when in use. In some instances, the embodiment of FIG. 4B may prevent light from a light source from being reflected off the inner or outer surface of the lens 135b back to the optical sensor. Finally, the lens 135c shown in FIG. 4C has a flat inner surface and a flat outer surface. In some embodiments the flat inner surface of lens 135c may be at a 90 degree angle relative to the longitudinal axis of the lens 135c or stylet body (120 of FIG. 4), or may be at a different angle such as 1-89 degrees. The particular shape of lens 135a, 135b, 135c used in connection with a particular sheath may depend on the nature of the treatment and characteristics (i.e., shape of lens, type of light source, type of optical sensor, and various coatings) of the medical instrument or lens.

FIG. 4 further illustrates a seal 137 disposed adjacent the proximal end of the sheath 130. The seal 137 may be configured to mate with, and seal against, the inside diameter of a component disposed around the sheath 130. For example, referring to FIGS. 1 and 4, the elongate tube 140 may comprise a proximal port 145 and a distal port 147. The seal 137 of the sheath 130 may interact with the proximal end 141 of the elongate tube 140, sealing the proximal end 141 of the elongate tube 140. Distal of the seal 137, there may be a gap between the outside diameter of the sheath 130 and the inside diameter of the elongate tube 140. This gap may comprise a flow path through the elongate tube assembly 100 from the proximal port 145 to the distal port 147. Because this flow path is outside the sheath 130, the flow path may be in fluid communication with the body environment without the stylet body 120 being in communication with the flow path or the body environment. An analogous flow path or gap is described and shown in connection with FIG. 7, discussed in detail below.

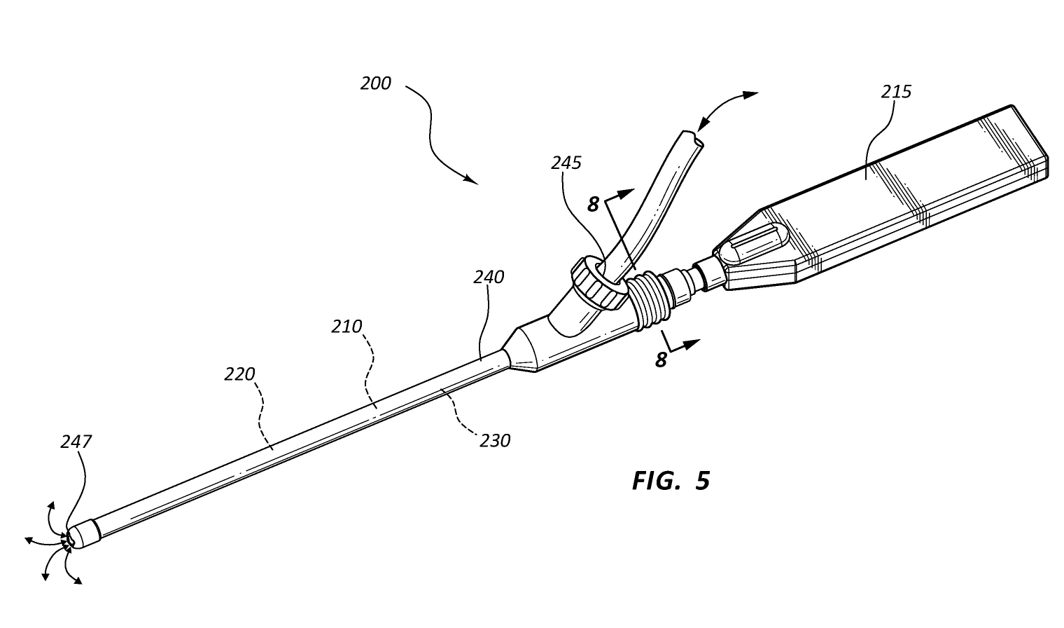

FIG. 5 is a perspective view of another embodiment of an elongate tube assembly 200 that can, in certain respects, resemble components of the elongate tube assembly 100 described in connection with FIGS. 1-4 above. It will be appreciated that all the illustrated embodiments may have analogous features. Accordingly, like features are designated with like reference numerals, with the leading digits incremented to “2.” (For instance, the elongate tube assembly is designated “100” in FIG. 1, and an analogous elongate tube assembly is designated as “200” in FIG. 5.) Relevant disclosure set forth above regarding similarly-identified features thus may not be repeated hereafter. Moreover, specific features of the elongate tube assembly 200 and related components shown in FIG. 5 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the elongate tube assembly 200 of FIG. 5. Any suitable combination of the features, and variations of the same, described with respect to the elongate tube assembly 100 and components illustrated in FIGS. 1-4 can be employed with the elongate tube assembly 200 and components of FIG. 5, and vice versa. This pattern of disclosure applies equally to further embodiments depicted in subsequent figures and described hereafter.

FIG. 5 is a perspective view of an elongate tube assembly 200 illustrating flow through the assembly 200. The elongate tube assembly 200 of FIG. 5 comprises a stylet body 220 having a connector 215, a sheath 230, and an elongate tube 240. In some embodiments, the elongate tube 240 may be sufficiently compliant such that it may be manipulated and/or displaced as the stylet body 220 is displaced. For example, the distal end of the stylet body 220 may be manipulated (for example, through use of a steering mechanism as described above) by user input via the connector 215. The elongate tube 240, disposed over the stylet body 220, may follow the stylet body 220, thus allowing a user to displace and position the elongate tube 240 by displacing the stylet body 220. Additionally, in some embodiments, the elongate tube 240 and stylet body 220 may be coupled such that rotation of the stylet body 220 about its longitudinal axis also rotates the elongate tube 240. For instance, a proximal seal, such as the seal 137 of FIG. 4, may be configured to couple the elongate tube 240 and stylet body 220 with respect to rotational movement.

In the embodiment of FIG. 5, the elongate tube 240 comprises a proximal port 245 and a distal port 247. As indicated by the arrows, flow into the proximal port 245 may result in flow out of the distal port 247 and vice versa. As described above, in connection with FIGS. 1 and 4, in some embodiments, there may be a gap between the outside diameter of the sheath 230 and the inside diameter of the elongate tube 240. This gap may, thus, create a flow path along the longitudinal direction of the elongate tube 240 and stylet body 220. The gap may be sealed at the proximal end (i.e., by a seal analogous to seal 137 of FIG. 4), thus creating a flow channel allowing fluid communication between the proximal end and the distal end of the stylet body 220 or elongate tube 240. The gap or flow path is further described and illustrated in connection with FIG. 7.

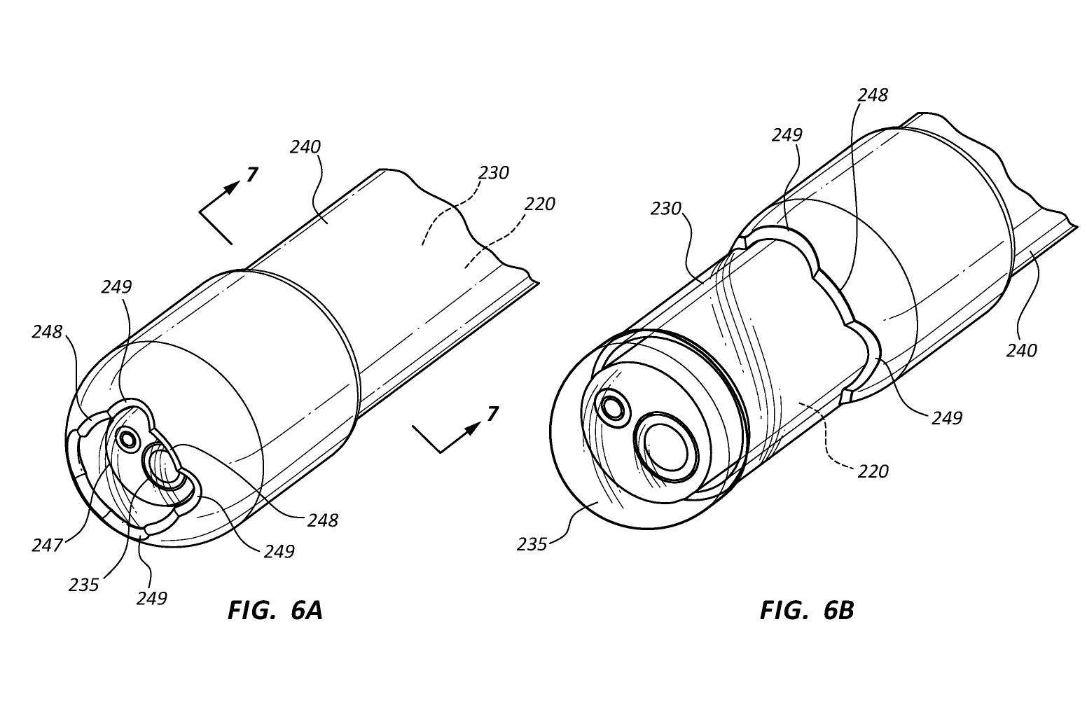

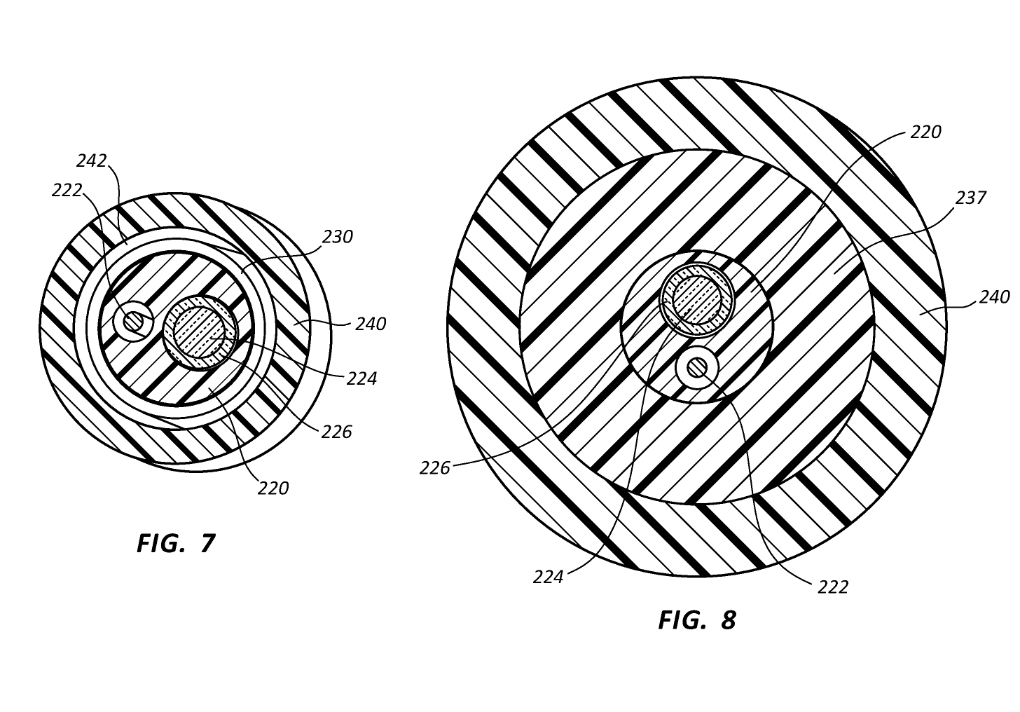

FIG. 6A is an enlarged view of the distal end of the elongate tube assembly 200 of FIG. 5 in a first configuration. In the embodiment of FIG. 6A, the distal port 247 of the elongate tube 240 comprises flutes 249 positioned about the opening of the distal port 247. Tabs 248 or protrusions may be positioned between each flute 249. In some embodiments, the stylet body 220 and sheath 230 may be axially displaceable with respect to the elongate tube 240. In the configuration shown in FIG. 6A, the stylet body 220 and sheath 230 are positioned such that the distal end of the sheath 230 provided around the distal end of the stylet body 220 is in contact with the tabs 248 of the distal port 247. The tabs 248 may be configured to provide resistance to further advancement of the stylet body 220 and sheath 230. Further, when the stylet body 220 and sheath 230 are in contact with the tabs 248, flow through the distal port 247 may be directed through the flutes 249, as the stylet body 220 and sheath 230 are positioned such that they tend to block or restrict flow through the center of the distal port 247. In other configurations, the distal ends of the stylet body 220 and sheath 230 may be positioned proximal to the distal port 247 such that they do not block flow through the center of the distal port 247.

Whether the stylet body 220 and sheath 230 are positioned at or proximal to the distal port 247, the tabs 248, flutes 249, or other features of the distal port 247 may be configured both to allow flow through the distal port 247 and to direct the flow. For example, the shape of the distal port 247 in connection with the tabs 248 and flutes 249 may tend to direct flow across the lens 235 of the sheath 230, acting to flush the environment adjacent the lens 235. Thus, flow out of the distal port 247 may be used to keep bodily structures or fluid from impeding light transfer across the lens 235. In other embodiments, additional tabs 248, projections, or other structures coupled to the device may be configured to direct flow to flush and/or clean the lens 235 during use.

FIG. 6B is an enlarged view of the distal end of the elongate tube assembly 200 of FIGS. 5 and 6A in a second configuration. In the configuration of FIG. 6B, the stylet body 220 and sheath 230 are axially extended beyond the distal port (247 of FIG. 6A) of the elongate tube 240. The tabs 248 may be configured to be sufficiently compliant to deform, as shown in FIG. 6B, to allow extension of the stylet body 220 and sheath 230. Further, the flutes 249 may be configured to allow flow even when the stylet body 220 and sheath 230 are so extended. The lens 235 of the sheath 230 is also shown in FIG. 6B.

FIG. 7 is a cross-sectional view of the elongate tube assembly 200 of FIG. 6A taken through plane 7-7. FIG. 7 illustrates the position of the gap or flow path 242 between the outside diameter of the sheath 230 and the inside diameter of the elongate tube 240. The stylet body 220, a steering cable 222, portions of light source optical fibers 226 and optical transmission optical fibers 224, and the sheath 230 are also shown.

In some embodiments, the flow path 242 may comprise an annular gap around the sheath 230. In other embodiments, the flow path 242 may not completely encircle the sheath 230, but rather be disposed around a portion of the sheath 230. For example, in some instances, the sheath 230 and stylet body 220 may be non-concentrically located within the elongate tube 240. Thus, the flow path 242 may only partially encircle the sheath 230, while still defined by the gap or space between the outside diameter of the sheath 230 and the inside diameter of the elongate tube 240. In some embodiments, the components may be disposed such that the sheath 230 may move within the elongate tube 240 during use, thus repositioning the relative position of the fluid flow path 242.

In other embodiments, the flow path 242 may comprise longitudinal grooves in the inside diameter of the elongate tube 240. These grooves may be provided in instances wherein the outside diameter of the sheath 230 is configured to contact the inside diameter of the elongate tube 240. In such instances, longitudinal grooves in the inside diameter of the elongate tube 240 may define gaps, or flow paths 242 disposed between the sheath 230 and the elongate tube 240. Further, in embodiments wherein the sheath 230 is smaller than the inside diameter of the elongate tube 240 (such that a gap is present between the components) the flow path 242 may comprise the gap in addition to longitudinal grooves. In some embodiments, longitudinal grooves may be aligned with features at the distal port (247 of FIGS. 6A and 6B), such as the flutes (249 of FIGS. 6A and 6B).

FIG. 8 is a cross-sectional view of the elongate tube assembly 200 of FIG. 5 taken through plane 8-8. FIG. 8 illustrates the interface of the seal 237 with the inside diameter of the elongate tube 240 adjacent the proximal end of the sheath (230 of FIG. 5). The stylet body 220, a steering cable 222, and portions of light source optical fibers 226 and optical transmission optical fibers 224 are also shown.

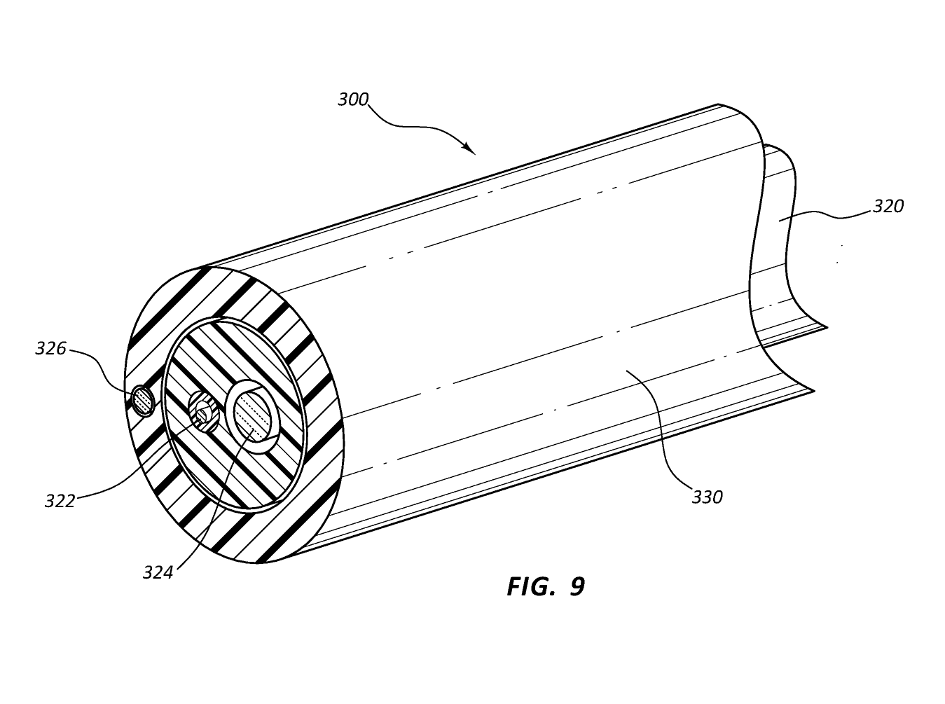

FIG. 9 is an enlarged perspective view of a portion of the distal end of another embodiment of an elongate medical instrument assembly 300. In the embodiment of FIG. 9, the instrument comprises a stylet body 320 incorporating a steering cable 322 and an optical sensor 324. The assembly 300 further comprises a sheath 330 disposed around the stylet body 320. For convenience in viewing the other components, no lens is shown, though any lens configuration disclosed herein, including a flat and/or compliant lens, may be used in connection with this embodiment. In the embodiment of FIG. 9, a light source 326 is disposed within a wall of the sheath 330. The light source 326 may comprise, for example, fiber optic strands configured to transmit and emit light. Positioning the light source 326 in this manner may eliminate distortion or “washing out” of the optical sensor 324 due to light reflection from the light source 326 off the inside surface of the lens. A light source 326 positioned in the wall of the sheath 330 may not be isolated from contact with bodily fluid, thus the light source 326 may be positioned such that it is in direct communication with the body environment. In some such embodiments, the light source 326 may be disposable and/or reusable with the sheath 330.

Furthermore, in other embodiments, the sheath 330 may be configured with other components disposed within the wall of the sheath 330. For example, flow path lumens, steering components, or other elements may be disposed within the wall of the sheath 330. Thus, in some embodiments, a lumen positioned in the wall of the sheath 330 (analogous in placement to the light source 326) may be used to provide a flow path from the proximal end of the device to the distal end. Such a lumen may be used in connection with, or in place of, a flow path outside the sheath 330, such as flow path 242 of FIG. 7. Thus, elongate medical devices comprising a stylet (110 of FIG. 1) and/or a sheath 330 may or may not be used in connection with another member, such as an elongate tube (240 of FIG. 5).

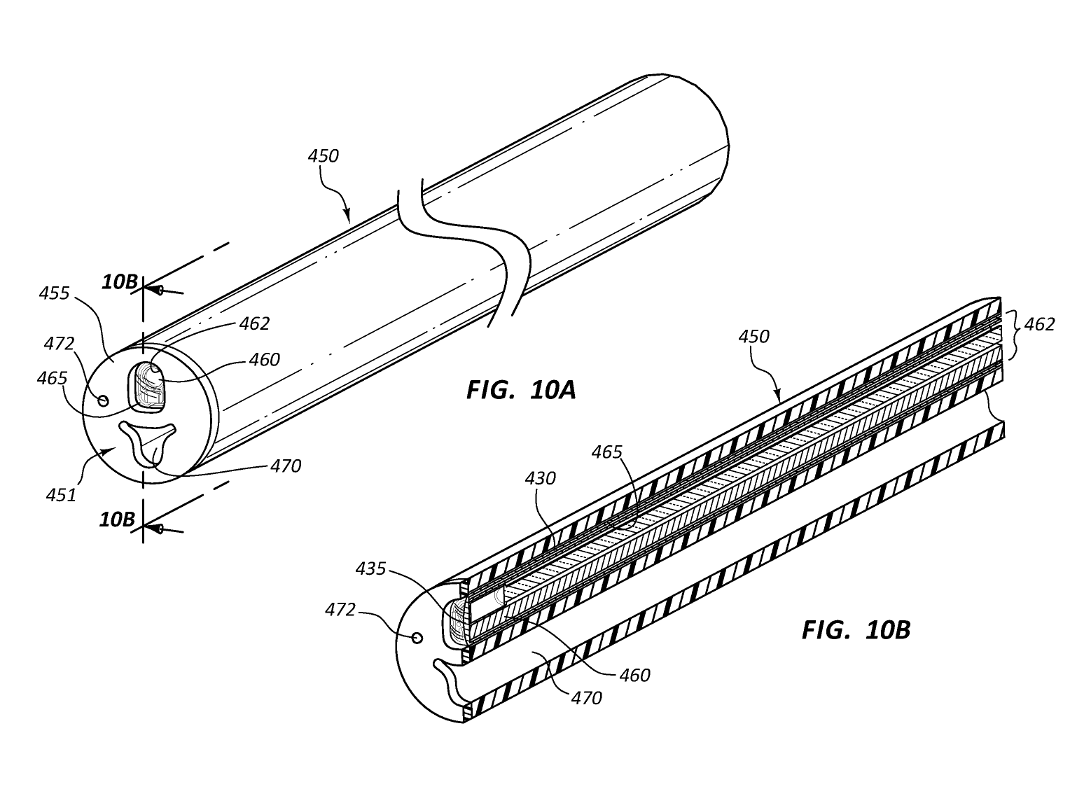

FIGS. 10A-21 illustrate various embodiments of endoscopes and components configured for use therewith. As used in the following description, the term “endoscope” is used broadly, to indicate any elongate instrument configured for imaging within the human body. Thus, endoscopes may or may not include additional components such as steering mechanisms or delivery lumens. Analogous to the stylet and/or other components described in embodiments above, endoscopes may be used in connection with sheaths to isolate certain components from interaction with bodily fluids. Further, in some embodiments, a fluid flow path may be provided around the sheath. In some embodiments the fluid flow path may be configured to flush a lens or other portion of the sheath.

FIG. 10A is a partial perspective view of a portion of an endoscope 450. The distal end 451 of the endoscope 450 may comprise a cap member 455. Further, an imaging component 460 may be disposed within a lumen or channel of the endoscope 450, such as imaging lumen 462. The imaging component 460, further described below, may include subcomponents such as an optical sensor and/or a light source. The endoscope 450 may further comprise a working lumen 470 configured to allow a practitioner to pass instruments, fluids, or other items from the proximal end of the endoscope 450 to a treatment location within the body. An aspirating lumen 472 may also be provided in connection with particular therapies. The working lumen 470 and aspirating lumen 472 may be in fluid or other communication with ports or openings at or adjacent the proximal end of the endoscope 450 to facilitate use of the endoscope 450 in treatments, including minimally invasive treatments.

FIG. 10B is a cross sectional view of the endoscope 450 of FIG. 10A. In the view of FIG. 10B, the working lumen 470 and the imaging component 460 are shown in cross section. The aspirating lumen 472 of the endoscope 450 can also be seen. The imaging component 460 may be disposed within an imaging lumen 462. The imaging component 460 may be displaceable within, and removable from, the imaging lumen 462. Thus, in some embodiments, the imaging component 460 may be configured to be reusable in connection with multiple endoscopes 450.

The imaging component 460 may be isolated from contact with the body environment by a sheath 430. The sheath 430 is analogous to any of the sheaths (130, 230, 330) disclosed in connection with FIGS. 1-9; disclosure provided in connection with any sheath may therefore be applicable to any other sheath. For example, the sheath 430 may be configured with a geometric or compliant lens 435 disposed adjacent the distal end of the sheath 430. The sheath 430 may be configured to obviate the need to sterilize the imaging component 460 for every procedure.

A fluid flow path 465 may be disposed around the sheath 430. In other words, a gap may be disposed between the sheath 430 and the inside of the imaging lumen 462. In some embodiments, the fluid flow path 465 may be in communication with a port or other input component adjacent the proximal end of the endoscope 450. Flow through the fluid flow path 465 may be configured to flush the lens 435 to facilitate viewing via the imaging component 460.

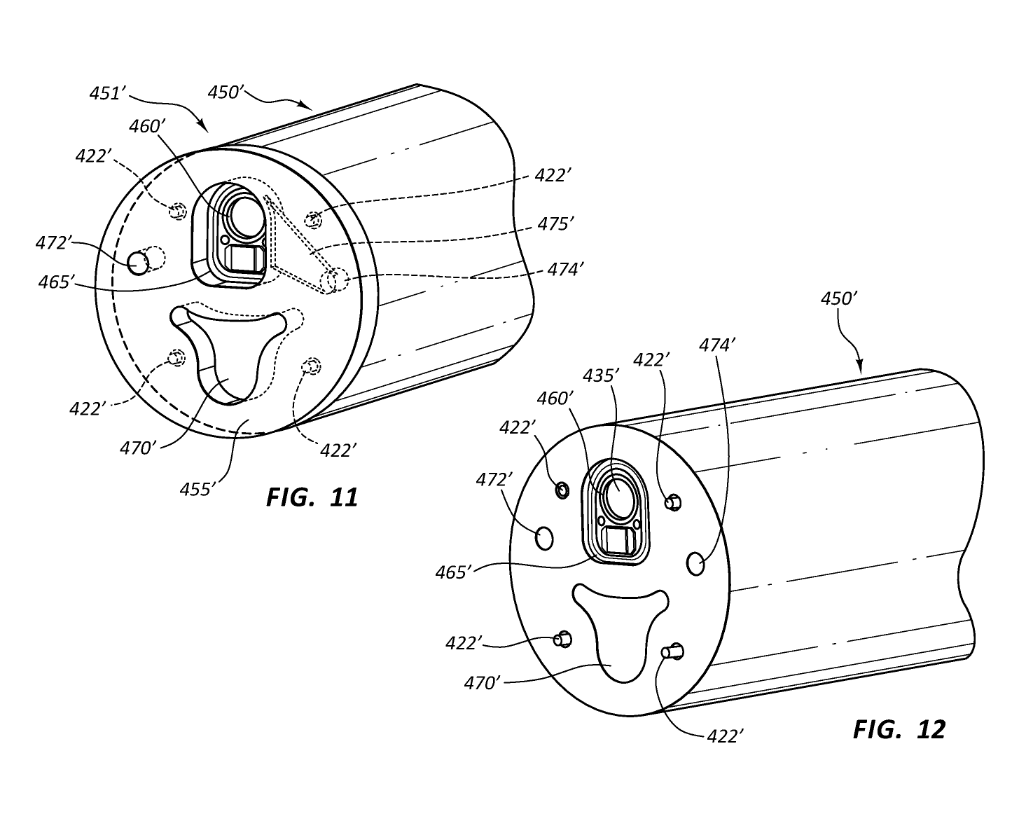

FIG. 11 is a partial perspective view of the distal end 451′ of an endoscope 450′. In the embodiment of FIG. 11, the endoscope 450′ is an alternative configuration of the endoscope 450 shown in FIGS. 10A and 10B. Thus, the two embodiments use corresponding reference numerals with the numerals of the latter embodiment designated by an apostrophe or “prime” indicator. An endoscope cap 455′ is illustrated, with certain components located under the endoscope cap 455′ shown in phantom lines. FIG. 12 is a partial perspective view of the endoscope 450′ of FIG. 11 with the endoscope cap 455′ removed. Referring to both of these figures, a working lumen 470′ and aspirating lumen 472′ are shown. Further, the endoscope 450′ comprises a flushing lumen 474′ and a fluid flow path 465′ disposed around an imaging component 460′. The flushing lumen 474′ may be used in connection with the fluid flow path 465′; in some embodiments, fluid may be circulated through these two channels to flush a lens 435′ component. A fluid control portion 475′ may be provided in connection with the endoscope cap 455′ to direct fluid from or to the flushing lumen 474′. In other embodiments, an endoscope 450′ may only have one of the flushing lumen 474′ and the fluid flow path 465′.

The endoscope 450′ of FIGS. 11 and 12 further comprises steering cables 422′ configured to direct the distal end 451′ of the endoscope 450′. In the illustrated embodiment, four steering cables 422′ are configured to work simultaneously to direct the endoscope 450′. In other embodiments, one, two, three, or more cables 422′ may be used.

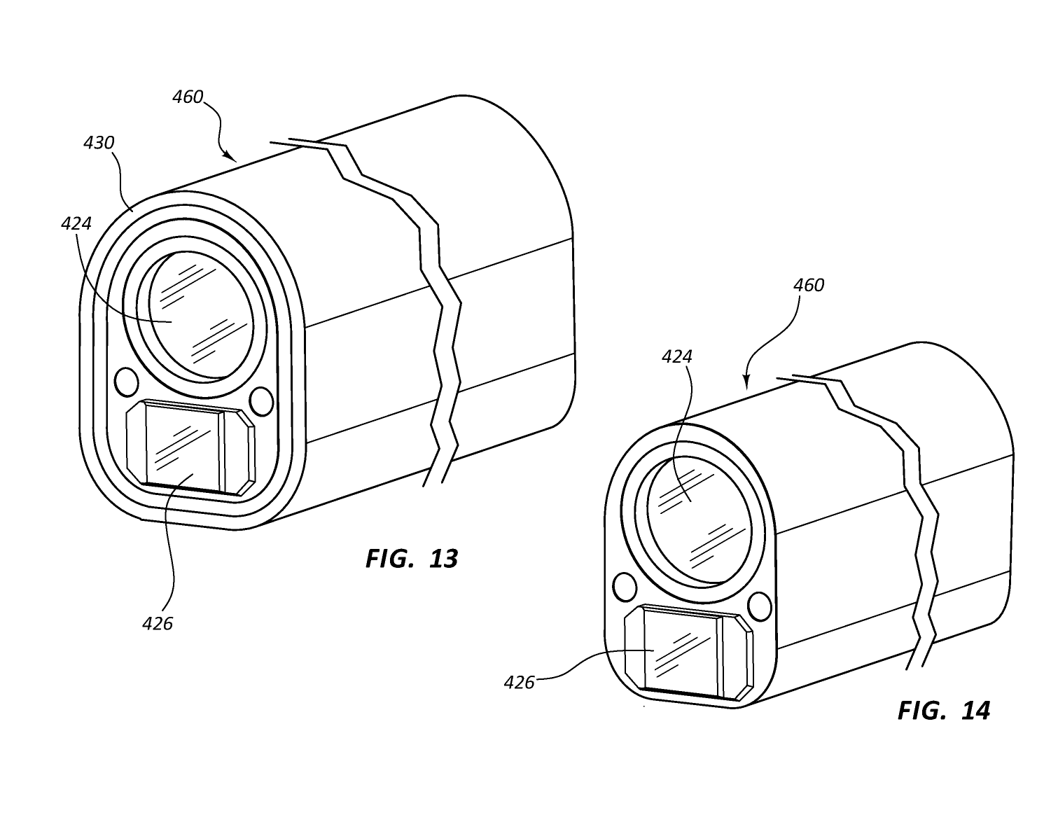

FIG. 13 is a perspective view of the imaging component 460 of the endoscope 450 of FIGS. 10A and 10B. FIG. 14 is a perspective view of the imaging component 460 of FIG. 13. The sheath 430 is shown in FIG. 13, though the lens (435 of FIG. 10B) is removed for convenience in FIG. 14.

Analogous to the embodiments described throughout, the imaging component 460 may include an optical sensor 424 and/or a light source 426. The optical sensor 424 may comprise fiber optical cables, a CCD or CMOS camera, and so forth. The light source 426 may also comprise fiber optical cables, or could comprise another light source 426 such as an LED.

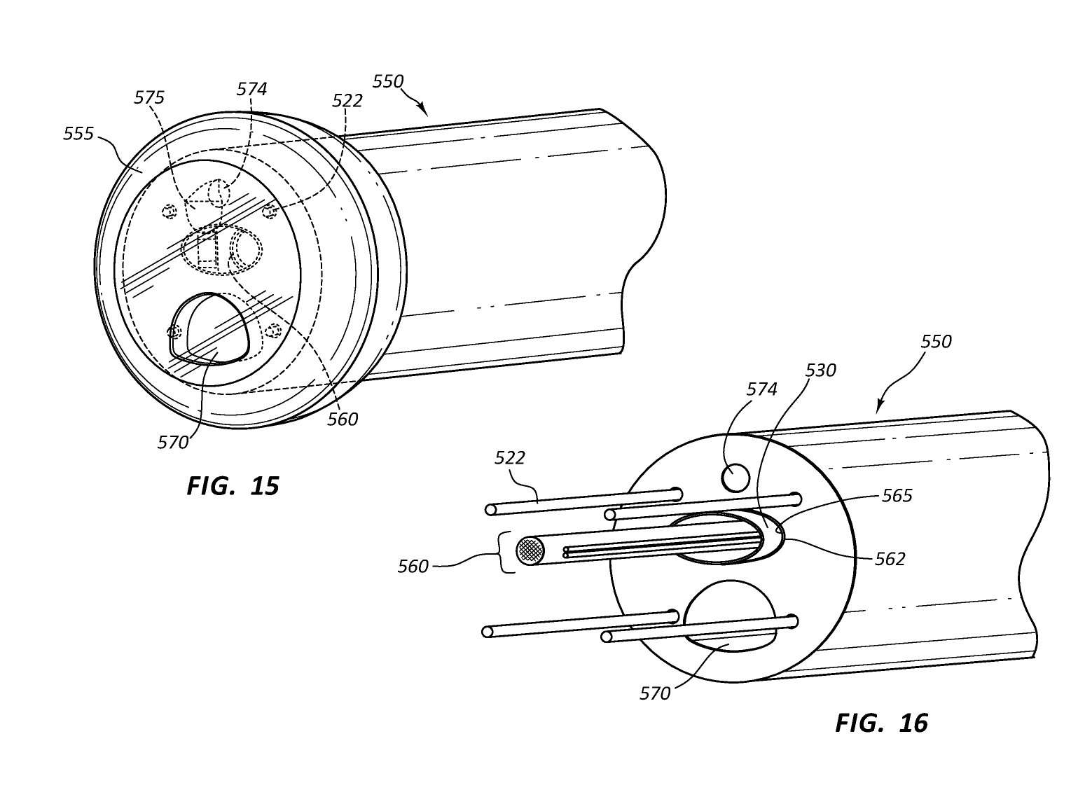

FIG. 15 is a perspective view of another embodiment of an endoscope 550 that can, in certain respects, resemble components of the endoscopes 450, 450′ described in connection with FIGS. 10A-14 above. As with the elongate tube embodiments described in connection with FIGS. 1-9, it will be appreciated that all the illustrated endoscope embodiments may have analogous features. Accordingly, like features are designated with like reference numerals, with the leading digits incremented to “5.” (For instance, the endoscope is designated “450” in FIG. 10A, and an analogous endoscope is designated as “550” in FIG. 15.) Relevant disclosure set forth above regarding similarly identified features thus may not be repeated hereafter. Moreover, specific features of the endoscope 550 and related components shown in FIG. 15 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the endoscope 550 of FIG. 15. Any suitable combination of the features, and variations of the same, described with respect to the endoscope 450, 450′ and components illustrated in FIGS. 10A-14 can be employed with the endoscope 550 and components of FIG. 15, and vice versa. This pattern of disclosure applies equally to further embodiments depicted in subsequent figures and described hereafter. Furthermore, any component described in connection with any embodiment, whether from FIGS. 1-9 (elongate tube embodiments) or subsequent figures (endoscope embodiments) may have analogous components in any other embodiment. Disclosure provided in connection with any embodiment is applicable to such analogues.

FIG. 15 is a partial perspective view of the distal end of an endoscope 550 and FIG. 16 is a partial cut-away view of the endoscope 550 of FIG. 15. The endoscope 550 comprises an endoscope cap 555 with a working lumen 570, which extends through the endoscope cap 555. Steering cables 522 are also provided in connection with this embodiment. The view of FIG. 16 illustrates a partial cut-away view of the endoscope body, illustrating the position of the steering cables 522 and the imaging component 560 within the endoscope 550. Further, a sheath 530 may be disposed over the imaging component 560 to isolate the imaging component 560 from contact with the body environment of a patient.

In the illustrated embodiment, a flushing lumen 574 is provided in connection with a fluid control portion 575. The flushing lumen 574 and fluid control portion 575 may be configured to direct fluid flow such that a portion of the sheath 530 adjacent the distal end of the imaging component 560 is flushed to facilitate viewing. In some embodiments, a fluid flow path 565 may be disposed between the sheath 530 and an imaging component channel 562. As with other embodiments, the fluid flow path 565 may be configured to provide flow to flush the distal end of the sheath 530, and in some embodiments, in connection with the flushing lumen 574.

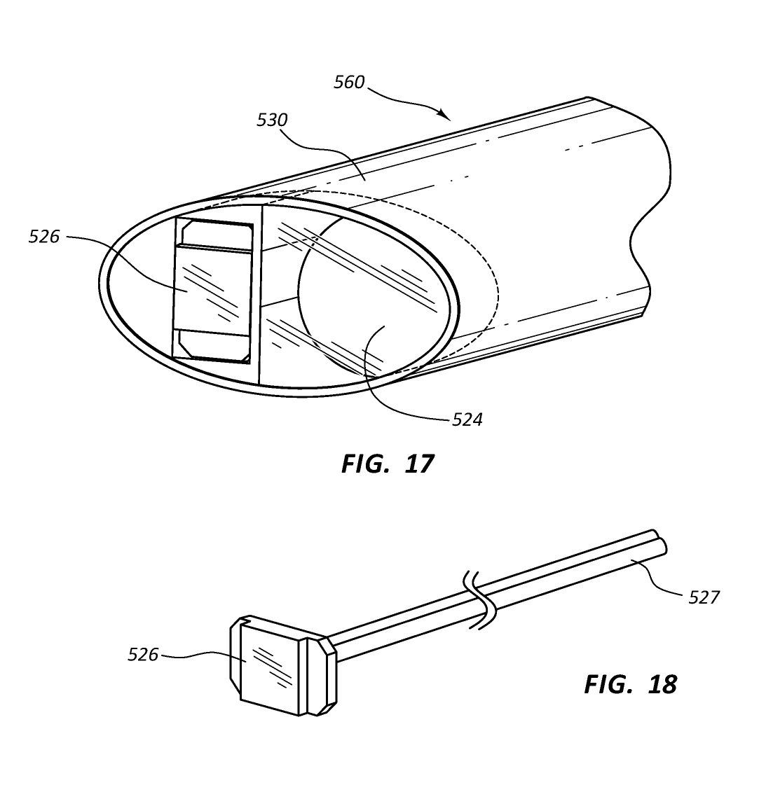

FIG. 17 is partial perspective view of the imaging component 560 of the endoscope 550 of FIG. 15. As in other embodiments, the imaging component 560 may be isolated by a sheath 530 disposed about the imaging component 560 to facilitate reuse of the imaging component 560 without sterilization between procedures. The imaging component 560 may comprise an optical sensor 524 and/or a light source 526. In some alternate embodiments, the light source 526 may be disposed within the sheath 530 and not isolated in the same manner as the optical sensor 524.

The light source 526 and optical sensor 524 may both comprise components with optical communication with the proximal end of the endoscope 550 (such as fiber optic cable) or comprise components configured for only electrical communication with the proximal end of the endoscope 550 (such as cameras and/or LEDs). For example, FIG. 18 is a perspective view of the light source 526 of the imaging component 560 of FIG. 17. In this embodiment, the light source 526 comprises an LED coupled to one or more electrical connections 527, such as wires.

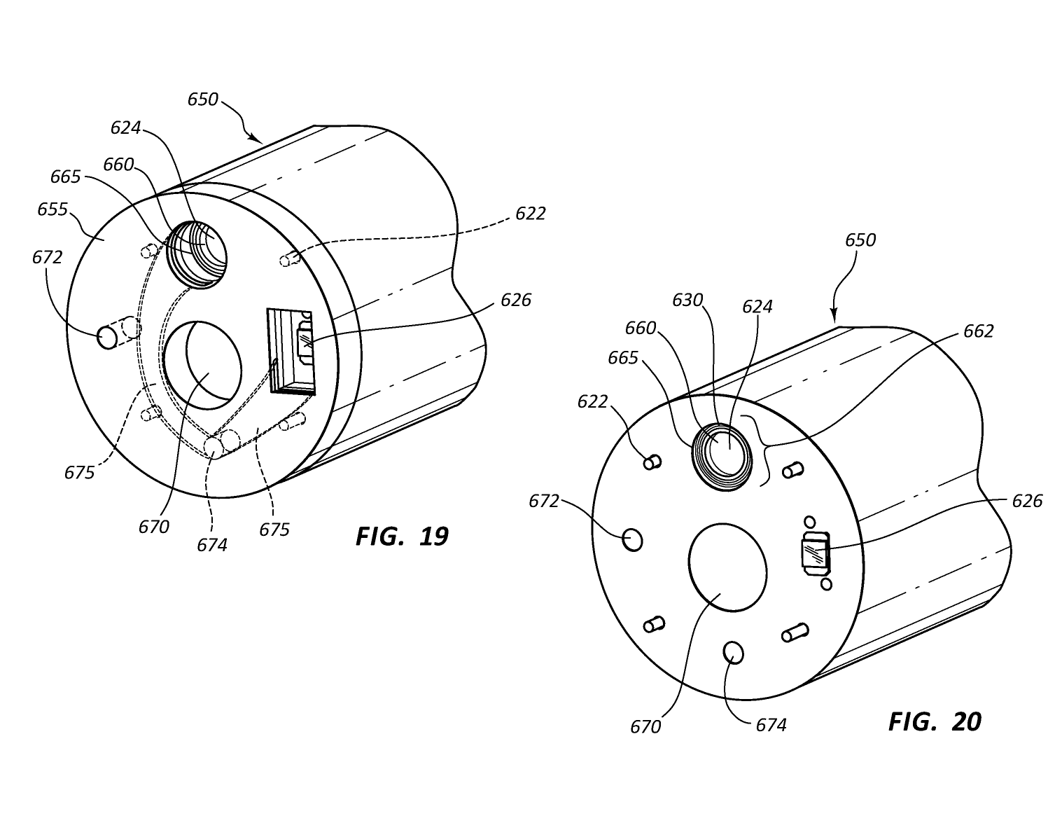

FIG. 19 is a partial perspective view of the distal end of an endoscope 650. FIG. 20 is a partial perspective view of the endoscope 650 of FIG. 19 with the endoscope cap 655 removed. The embodiment of these figures includes a working lumen 670, steering cables 622, an aspirating lumen 672, and a flushing lumen 674. Furthermore, in the illustrated embodiment, an imaging component 660 comprising an optical sensor 624 is provided separately from a light source 626. The imaging component 660 may be isolated from the body by a sheath 630 while the light source 626 may or may not be isolated. In some embodiments, the endoscope cap 655 may be configured with a transparent portion, such as a glass portion, directly adjacent the light source 626 to facilitate light transmission across the endoscope cap 655. Fluid directing portions 675 are provided in connection with the flushing lumen 674 and may be configured to direct flow toward both the light source 626 and the imaging component 660. In some embodiments, a fluid flow path 665 may be disposed between the sheath 630 and an imaging lumen 662 to flush the distal end of the sheath 630 in connection with the flushing lumen 674.

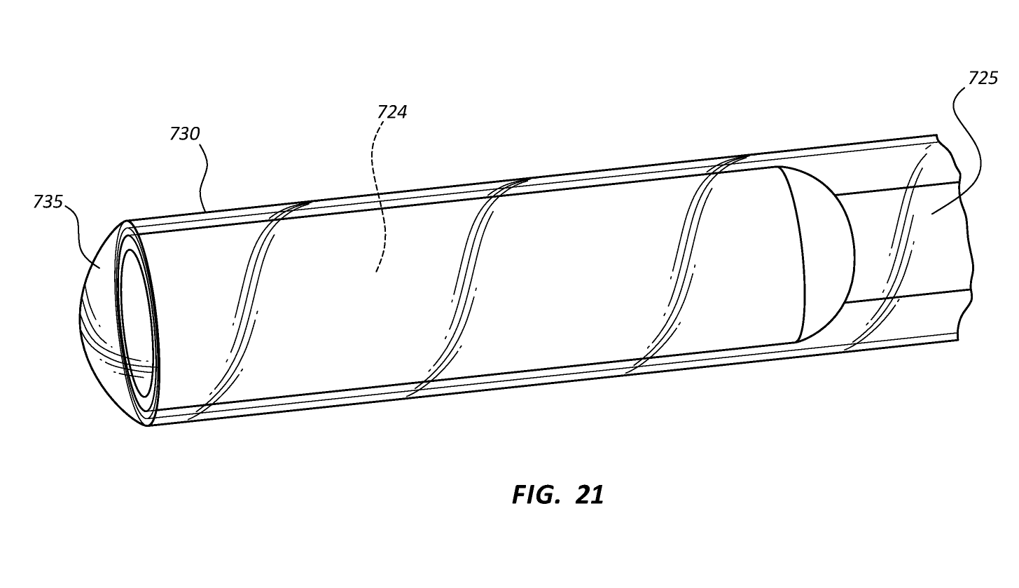

FIG. 21 is a side view of a camera 724 and sheath 730. A camera 724, such as a CMOS or CCD camera, may be configured for use as an imaging component (660 of FIGS. 19 and 20), in any of the above embodiments. The camera 724 may be isolated from contact with bodily fluids by the sheath 730, facilitating reuse of the camera 724 without resterilization. The camera 724 may be in communication with the proximal end of a device through use of connections 725, such as electrical connections or wires. The sheath 730 may comprise a lens 735 configured to facilitate light transmission across the sheath 730 to the camera 724. In some embodiments, the lens 735 may be formed in a particular shape, such as the partially domed shape of lens 735. In other embodiments, the lens 735 may comprise an elastic or complaint material configured to stretch over the distal end of the camera 724.

Without further elaboration, it is believed that one skilled in the art can use the preceding description to utilize the present disclosure to its fullest extent. The examples and embodiments disclosed herein are to be construed as merely illustrative and exemplary and not as a limitation of the scope of the present disclosure in any way. It will be apparent to those having skill in the art, and having the benefit of this disclosure, that changes may be made to the details of the above-described embodiments without departing from the underlying principles of the disclosure herein.

1. An elongate medical instrument comprising:
a body member having a proximal end and a distal end, the body member comprising,
a steering mechanism and
an image sensing component;
a connector component coupled to the proximal end of the body member;
a light emitting component; and
a removable sheath configured to be disposed around the body member such that the sheath isolates the body member from communication with bodily fluids; and
a fluid flow path disposed around at least a portion of the removable sheath.

2. The elongate medical instrument of claim 1, wherein the removable sheath comprises a lens portion, the lens portion configured to allow transmission of light across the lens portion.

3. The elongate medical instrument of claim 2, wherein the lens portion comprises at least one of a geometrically-shaped interior surface and a geometrically-shaped exterior surface.

4. The elongate medical instrument of claim 2, wherein the fluid flow path provides fluid communication between a proximal port disposed adjacent the connector component and a distal port disposed adjacent the distal end of the body member, and wherein the removable sheath isolates the body member from the fluid flow path.

5. The elongate medical instrument of claim 4, wherein the fluid flow path is configured to direct fluid from the distal port such that the fluid flushes the lens portion of the removable sheath.

6. The elongate medical instrument of claim 1, wherein the light emitting component is disposed within a wall of the removable sheath.

7. The elongate medical instrument of claim 1, wherein the light emitting component is disposed within the body member.

8. The elongate medical instrument of claim 1, wherein the image sensing component comprises at least one of: fiber optic strands, a CCD camera, and a CMOS camera.

9. The elongate medical instrument of claim 1, wherein the steering mechanism comprises at least one steering cable.

10. The elongate medical instrument of claim 1, further comprising an elongate tube disposed around the removable sheath, the elongate tube having a proximal end and a distal end.

11. The elongate medical instrument of claim 10, wherein the fluid flow path is positioned between the elongate tube and the removable sheath.

12. The elongate medical instrument of claim 11, wherein the elongate tube comprises flutes adjacent the proximal end of the elongate tube, the flutes configured to allow fluid flow from the fluid flow path when the body member is extended beyond the distal end of the elongate tube.

13. The elongate medical instrument of claim 12, further comprising tabs disposed between the flutes, the tabs configured to engage the distal end of the body member such that the tabs exert a locking force configured to inhibit extension of the body member from the elongate tube.

14. The elongate medical instrument of claim 13, wherein the tabs are configured to allow extension of the body member from the elongate tube in response to a distally-oriented force on the body member, such that the distally-oriented force overcomes the locking force.

15. The elongate medical instrument of claim 13, wherein the flutes and tabs are configured to direct fluid flow from the fluid flow path such that the fluid flow flushes a portion of the removable sheath.

16. The elongate medical instrument of claim 1, wherein the elongate medical instrument comprises one of: a J-type feeding tube, a G-type feeding tube, a G-J extension-type feeding tube, an NG-type feeding tube, an NJ-type feeding tube, a J-type drug delivery tube, an NJ-type drug delivery tube, an NG-type drug delivery tube, a G-type drug delivery tube, and an G-J extension-type drug delivery tube.

17. An elongate medical instrument comprising:
a body member having a proximal end and a distal end, the body member comprising,
a steering mechanism and
an image sensing component;
a connector component coupled to the proximal end of the body member;
a light emitting component; and
a removable sheath configured to be disposed around the body member such that the removable sheath isolates the body member from communication with bodily fluids; and
an elongate tube disposed around the removable sheath, the elongate tube having a proximal end and a distal end.

18. The elongate medical instrument of claim 17, further comprising a fluid flow path disposed between the removable sheath and the elongate tube.

19. The elongate medical instrument of claim 18, wherein fluid flow through the fluid flow path is configured to flush a portion of the removable sheath.

20. The elongate medical instrument of claim 17, wherein the fluid flow path comprises a plurality of longitudinally-oriented grooves in an inside diameter of the elongate tube.

21. The elongate medical instrument of claim 20, wherein the elongate tube comprises flutes adjacent the distal end of the elongate tube, the flutes configured to allow fluid flow from the fluid flow path when the body member is extended beyond the distal end of the elongate tube.

22. The elongate medical instrument of claim 21, further comprising tabs disposed between the flutes, the tabs configured to engage the distal end of the body member such that the tabs exert a locking force configured to inhibit extension of the body member from the elongate tube.

23. The elongate medical instrument of claim 22, wherein the tabs are configured to allow extension of the body member from the elongate tube in response to a distally-oriented force on the body member, such that the distally-oriented force overcomes the locking force.

24. A method of introducing an elongate medical instrument into a patient's body, comprising:
obtaining an elongate medical instrument;
obtaining a removable sheath comprising a lens portion;
introducing the elongate medical instrument into the body such that the removable sheath isolates the elongate medical instrument from fluids within the patient's body; and
introducing flow through a fluid flow path disposed around the removable sheath such that the flow flushes the lens portion.

25. The method of claim 24, further comprising removing the elongate medical instrument and removable sheath from the patient's body.

26. The method of claim 24, further comprising:
inserting the elongate medical instrument and the sheath into an elongate tube prior to introducing the elongate medical instrument into the body;
positioning the elongate tube within the patient's body; and
removing the elongate medical instrument and sheath from the patient's body.

27. The method of claim 26, wherein the fluid flow path is disposed between the removable sheath and the elongate tube.
